(19) United States
(12) Patent Application Publication
(10) Pub. No.: US 2010/0105053 A1
(43) Pub. Date: Apr. 29, 2010

(54) APTAMER BASED SENSORS AND RELATED METHODS AND SYSTEMS

(76) Inventors: Hansang CHO, Albany, CA (US); Stephen M. LANE, Oakland, CA (US); Brian R. BAKER, Livemore, CA (US); Sebastian WACHSMANN-HOGIU, Sacramento, CA (US); Cynthia V. PAGBA, Sacramento, CA (US); Jeffrey B.H. TOK, Stanford, CA (US); Ted Alfred LAURENCE, Livermore, CA (US); Luke P. LEE, Orinda, CA (US)

Correspondence Address: LLNL/Steinfl & Bruno;John H. Lee, Assistant Laboratory Counsel, Lawrence Livermore National Laboratory, L-703, P.O. Box 808, Livermore, CA, 94551, US

(21) Appl. No.: 12/576,206

(22) Filed: Oct. 8, 2009

Related U.S. Application Data
(60) Provisional application No. 61/104,627, filed on Oct. 10, 2008.
(60) Provisional application No. 61/175,822, filed on May 6, 2009.

(51) Int. Cl. G01N 33/53; C12Q 1/68

(52) U.S. Cl. 435/6; 435/7.1

(57) ABSTRACT

An aptamer based-sensor comprising: a target binding aptamer attaching a Raman probe and a metal coated surface; and related methods and systems

### CROSS REFERENCE TO RELATED APPLICATIONS

This application claims priority to U.S. Provisional Application entitled “Aptamer-based SERRS Sensor for Specific, Sensitive, and Stable Detection” Ser. No. 61/104,627, filed on Oct. 10, 2008 Docket No. IL-12034, and to U.S. Provisional Application entitled “Integrated Microfluidic Platform With Nanoplasmonic Aptasensor For On-Chip Label-Free VEGF Detection In Dynamic Tumor Microenvironment” Ser. No. 61/175,822, filed on May 6, 2009, the disclosure of each of which is incorporated herein by reference in its entirety. The application may be further related to U.S. patent application entitled “Microfluidic Platform and Related Methods and Systems” Ser. No. to be assigned filed on the same day of the present application with Docket No. IL12098, which is also herein incorporated by reference in its entirety.

### STATEMENT OF GOVERNMENT GRANT

The United States Government has rights in this invention pursuant to Contract No. Contract No. DE-AC52-07NA27344 between the U.S. Department of Energy and Lawrence Livermore National Security, LLC, for the operation of Lawrence Livermore National Security.

### TECHNICAL FIELD

The present disclosure relates to aptamer-based sensors and related methods and systems.

### BACKGROUND

Aptamers, and in particular single-stranded (ss) oligonucleotides or peptides, are molecules used as diagnostic reagents and potential antibody replacements for the development of biomolecular nanosensors, due to their high affinity, specificity, and stability for analytes of interest.

Numerous aptamer-based sensors (aptasensors or aptamer beacons) have been developed to monitor the interaction with targets by measuring electron transfer, color change, or fluorescence quenching occurring following the aptamer binding to the target.

In view of the features of current aptamer-based sensors, achievement of specificity, sensitivity, and stability of the aptamer-based sensors with current approach can often be challenging.

### SUMMARY

Provided herein, are aptamer-based sensors, related methods, and systems that in several embodiments allow sensitive, specific, and reliable detection of a target of interest with a nanosensor that for size and simplicity is highly practical and manageable.

According to a first aspect, an aptamer based-sensor is described. The aptamer-based sensor herein described comprises: a target binding aptamer attaching a spectroscopic probe and a surface configured for detection through spectroscopic analysis. In the aptamer based sensor herein described the target binding aptamer attaching the spectroscopic probe is capable of specifically binding a corresponding target and is detectable in connection with the surface through surface enhanced spectroscopy of the surface. In several embodiments, the target binding aptamer is attached to the spectroscopic probe through direct covalent binding.

According to a second aspect, a method to detect a target is described. The method comprises detecting a first surface enhanced spectrum of a surface configured for detection through spectroscopic analysis, the surface presenting a target binding aptamer specific for the target, the target binding aptamer attaching a spectroscopic probe. The method further comprises contacting the target binding aptamer on the surface with the target for a time and under conditions to allow binding of the target with the target binding aptamer. The method also comprises detecting a second surface enhanced spectrum of the surface following contacting of the target binding aptamer with the target and comparing the first spectrum and the second spectrum. In several embodiments, a difference between the first spectrum and the second spectrum will be used to detect qualitatively or quantitatively the target through detection of the target binding aptamers that are not presented on the surface anymore following contact of the aptamer with the target.

According to a third aspect, a system to detect a target is described. The system comprises at least two of a target binding aptamer, a spectroscopic probe and a surface configured for detection through spectroscopic analysis. In the system, the target binding aptamer, the Raman probe and the surface are used in combination to detect the target according to a method herein described.

According to a fourth aspect, a method to manufacture an aptamer-based sensor is described. The method comprises; contacting a surface configured for detection through spectroscopic analysis with a target binding aptamer attaching a spectroscopic probe for a time and under condition to allow adsorption of the target binding aptamer on the surface.

The aptasensors methods and systems herein described can be used in several embodiments for sensitive and specific detection of targets, such as biomarkers, present in a solution contacted with the aptasensor and/or secreted by cells or other biofluids (such as serum, plasma, and saliva) operably connected to the aptasensors.

In particular, in several embodiments the aptasensors methods and systems herein described can be used to perform a quantitative detection on targets such as growth factors involved in signaling and additional pathways of biological relevance.

Additionally, in several embodiments the flexibility to comprise aptasensors and systems herein described can accommodate numerous and diverse target-specific aptamers with a variety of Raman probes multiplexed detection of targets.

Furthermore, the stability of aptasensors and systems herein described can be used in several embodiments for on-chip detection of growth factors secreted by cells performed in harsh cell culturing conditions (e.g. about 37° C. for the period longer than 1 week), at which antibodies cannot maintain the functionality.

Additionally, in several embodiments the simplicity of manufacturing aptasensors methods and systems herein described can be used in connection with microfluidics platforms for a simple and efficient detection of target in a microfluidic device through performance of low cost protein assays.

In several embodiments the aptasensors methods and systems herein described can be used in connection with any applications wherein qualitative or quantitative target detection is desirable, including but not limited to drug screening, biomedical diagnostics, illicit drug or bio-agent detection, in fields such as biotechnology medicine forensics, and environmental sensing.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the present disclosure and, together with the detailed description and example sections, serve to explain the principles and implementations of the disclosure.

FIG. 1 shows a schematic illustration of a fabricated SERRS aptasensor and detection of a target according to some embodiments herein described. In particular, in the illustration of FIG. 1c GNP-substrate is prepared by fixing citrate-coated GNPs on an APTES-treated glass substrate. In the schematics of FIGS. 1a and 1d, an unfolded a thiol-modified single-stranded Thrombin Binding Aptamer (TBA) attached to the Raman probe Methylene Blue (MB), is naturally unfolded in the absence of human α-thrombin protein, a target molecule. The unfolded TBA is physisorbed surface through electrostatic interactions or chemisorbed through gold-thiol bonds on gold nanoparticles (GNP) surface. As a large number of MB is present in the hot spots, where local surface plasmon resonance is induced, a strong surface enhanced resonance Raman scattering (SERRS) signal for MB is observed. In the illustration of FIGS. 1b and 1e in presence of thrombin, TBA is displaced from the surface, resulting in the prompt decrease of the SERRS signal.

FIG. 2 shows diagrams illustrating target detection performed with SERRS aptasensors according to some embodiments herein described. In particular, FIG. 2a shows a chart illustrating SERRS intensity of MB decreased with the increasing thrombin concentration. The inset shows a SEM image of aggregated GNPs and the scale bar resents 200 nm. FIG. 2b shows a chart illustrating a drop of the peak intensity at 1622 cm-1 of about 10% with 100 pM thrombin, and a peak intensity that reached the 50% saturating level with 1 μM thrombin. FIG. 2c shows a diagram illustrating proportionality of the SERRS signal drop to the thrombin concentration and a saturation level at 1 μM. Scale bar in an inset is 200 nm. The y-axis (normalized intensity (I/AU)), was plotted as a ratio of the measured intensity at each protein concentration over the intensity without thrombin in 1× thrombin binding buffer solution. Data shown represents the mean, with standard deviation, of four separate measurements.

FIG. 3 shows a diagram illustrating characterization of the specificity of aptamer-based SERRS detection method according to some embodiments herein described. Note that the y-axis, i.e. normalized intensity (I/AU), was plotted as a ratio of the measured intensity at each protein concentration over the intensity without thrombin in 1× thrombin binding buffer solution. Data shown represents the mean, with standard deviation, of four separate measurements.

FIG. 4 shows a diagram illustrating evaluation of stability of the developed method and the aptamer-based SERRS nanosensor in serum solution according to some embodiments herein described. The y-axis (normalized intensity (I/I 10% serum)), was plotted as a ratio of the measured intensity at each protein concentration over the intensity without thrombin in 10% serum thrombin binding buffer solution. Data shown represents the mean, with standard deviation, of four separate measurements

FIG. 5 shows a diagram illustrating evaluation of stability of the developed method and the aptamer-based SERRS nanosensor in harsh conditions according to some embodiments herein described. The y-axis (i.e. normalized intensity (I/AU)), was plotted as a ratio of the measured intensity at each protein concentration over the intensity in 1× thrombin binding buffer solution. Data shown represents the mean, with standard deviation, of four separate measurements.

### DETAILED DESCRIPTION

Provided herein are aptamer-based sensors for target detection performed through Surface Enhanced Resonance Raman Spectroscopy (SERRS) or other Surface Enhanced Spectroscopy.

The term “aptamers” as used here indicates oligonucleic acid or peptide molecules that are capable to bind a specific target.

The terms “oligonucleic acid”, “nucleotidic oligomer” or “oligonucleotide” as used herein, indicate an organic polymer composed of two or more monomers including nucleotides, nucleosides or analogs of three or more residues typically of 100 nucleotides or less. The term “nucleotide” refers to any of several compounds that consist of a ribose or deoxyribose sugar joined to a purine or pyrimidine base and to a phosphate group and that is the basic structural unit of nucleic acids. The term “nucleoside” refers to a compound (such as guanosine or adenosine) that consists of a purine or pyrimidine base combined with deoxyribose or ribose and is found especially in nucleic acids. The term “nucleotide analog” or “nucleoside analog” refers respectively to a nucleotide or nucleoside in which one or more individual atoms have been replaced with a different atom or a with a different functional group.

The terms “peptide” and “oligopeptide” as used herein indicate an organic linear, circular, or branched polymer composed of two or more amino acid monomers and/or analogs thereof with 50 or less amino acid monomers. As used herein the term “amino acid”, “amino acidic monomer”, or “amino acid residue” refers to any of the twenty naturally occurring amino acids, non-natural amino acids, and artificial amino acids and includes both D an L optical isomers. In particular, non-natural amino acids include D-stereoisomers of naturally occurring amino acids (these including useful ligand building blocks because they are not susceptible to enzymatic degradation). The term “artificial amino acids” indicate molecules that can be readily coupled together using standard amino acid coupling chemistry, but with molecular structures that do not resemble the naturally occurring amino acids. The term “amino acid analog” refers to an amino acid in which one or more individual atoms have been replaced, either with a different atom, isotope, or with a different functional group but is otherwise identical to original amino acid from which the analog is derived. All of these amino acids can be synthetically incorporated into a peptide or polypeptide using standard amino acid coupling chemistries.

In particular, aptamers in the sense of the present disclosure comprise single-stranded (ss) oligonucleotides and chemically synthesized peptides that have been engineered through repeated rounds of in vitro selection, or equivalent techniques identifiable by a skilled person, to bind to various targets.

The term “sensor” as used herein indicates a device that measures a physical quantity and converts it into a signal which can be read by an observer or by an instrument. For accuracy, all sensors need to be calibrated against known standards.

Accordingly, the wording “aptamer-based sensor”, aptasensor, or aptamer beacon used herein indicate a sensor that can be used to capture a target exploiting the affinity of aptamer to the target and that can be detected using techniques identifiable by a skilled person upon reading of the present disclosure.

The term “detect” or “detection” as used herein indicates the determination of the existence, presence or fact of a target or signal in a limited portion of space, including but not limited to a sample, a reaction mixture, a molecular complex and a substrate including a platform and an array. Detection is “quantitative” when it refers, relates to, or involves the measurement of quantity or amount of the target or signal (also referred as quantitation), which includes but is not limited to any analysis designed to determine the amounts or proportions of the target or signal. Detection is “qualitative” when it refers, relates to, or involves identification of a quality or kind of the target or signal in terms of relative abundance to another target or signal, which is not quantified. An “optical detection” indicates detection performed through visually detectable signals: spectra or images from a target of interest or a probe attached to the target.

The term “target” as used herein indicates an analyte of interest. The term “analyte” refers to a substance, compound or component whose presence or absence in a sample has to be detected. Analytes include but are not limited to biomolecules and in particular biomarkers. The term “biomolecule” as used herein indicates a substance compound or component associated to a biological environment including but not limited to sugars, amino acids, peptides proteins, oligonucleotides, polynucleotides, polypeptides, organic molecules, haptens, epitopes, biological cells, parts of biological cells, vitamins, hormones and the like. The term “biomarker” indicates a biomolecule that is associated with a specific state of a biological environment including but not limited to a phase of cellular cycle, health and disease state. The presence, absence, reduction, upregulation of the biomarker is associated with and is indicative of a particular state. The term “biological environment” refers to any biological setting, including, for example, ecosystems, orders, families, genera, species, subspecies, organisms, tissues, cells, viruses, organelles, cellular substructures, prions, and samples of biological origin. Exemplary targets comprise molecular targets such as small molecules, proteins, nucleic acids, and also cells, tissues and organisms.

The aptamer-based sensors of the present disclosure are detectable through spectroscopic detection techniques such as SERRS, SERS or SEF (herein collectively Surface Enhanced Spectroscopy). The term “Surface Enhanced Spectroscopy” as used herein indicates signal enhancement techniques where signal detection from corresponding spectroscopic probes is performed in connection with a metal surface. Exemplary spectroscopic techniques suitable to detect aptamer based sensor herein described comprise including Surface-Enhanced Resonance Raman Spectroscopy (SERRS), Surface-Enhanced Raman Spectroscopy (SERS), Surface-Enhanced Fluorescence (SEF), Surface-Enhanced Infrared Absorption (SEIRA), Surface-Enhanced Hyper-Raman Scattering (SEHRS), Surface-Enhanced Coherent Anti-Stokes Raman Scattering (SECARS), and additional techniques identifiable by a skilled person.

The term “spectroscopic probe” as used herein indicates any substance that is suitable to be detected based on an interaction between a radiation and the substance through a spectroscopic instrument. Exemplary spectroscopic probes comprise Raman probes and fluorescence probes. The terms “Raman active molecule” or “Raman probe” as used herein refer to a molecule capable having a polarization-dependant vibrational mode excited by an incident light. The vibrational energy stored in the molecule is transformed into a scattering light corresponding to a specific frequency. In particular, detected signals emitted by Raman probes can take the form of Raman spectra. Accordingly, in Raman spectra for a certain Raman probe, each peak represents the vibrational frequency corresponding to resonance energy of the functional groups in the Raman probe as detected. Therefore, Raman spectra are intrinsic properties of the molecules such as a “molecular fingerprint” to identify the molecule without need to use of any additional labels.

In some embodiments, Raman probes suitable to be included in the aptamer-based sensors herein described comprise Raman-active molecules having polarization-dependant translational and/or rotational modes. Exemplary Raman probes suitable to be used for aptamers based sensors herein described comprise Trans-1,2 bis-(4-pyridyl) ethylene (BPE), Cy-3, Cy-3.5, Cy-5, Cy-5.5, Cy-7, Rhodamine 6G (R6G), methylene blue (MB), 5-carboxyfluorescein or 6-carboxyfluorescein (FAM), N,N,N′,N′-tetramethyl-6-carboxyrhodamine (TAMRA), 6-carboxy-4,7,2′,7′-tetrachlorofluorescein (TET), 6-carboxy-Xrhodamine (ROX), (3-(5,6,4′,7′-tetrachloro-5′-methyl-3′,6′-dipivaloylfluorescein-2-yl)-propanamidohexyl-1-O-(2-cyanoethyl)-(N,N-diisopropyl)) Yakima Yellow®, 6-(((4(4,4-difluoro-5-(2-thienyl)-4-bora-3a,4a-diaza-s-indacene-3-yl)phenoxy)acetyl)amino)hexanoic acid (BODIPY TR-X) and additional probes identifiable by a skilled person upon reading of the present disclosure

The term “fluorescent probe” as used herein indicates any substance that is detectable through emission of a visible light by the substance following absorption by the same substance of light of a differing, usually invisible, wavelength. Exemplary fluorescent probes suitable in the aptamer-based sensors herein described comprise Cy-3, Cy-3.5, Cy-5, Cy-5.5, Cy-7, Rhodamine 6G (R6G), methylene blue (MB), TAMRA and additional probes identifiable by a skilled person.

In particular in several embodiments, the aptamer-based sensors herein described comprise either a Raman probe and/or a fluorescent probe and are detectable through surface plasmon enhanced spectroscopy

In several embodiments, spectroscopic probes used in the aptamer-based sensor of the present disclosure enable an enhancement factor e as much as 10¹⁴-10¹⁶, which allows the technique to be sensitive enough to detect single molecules.

In aptamers-based sensor herein described a spectroscopic probe is attached to a target binding aptamer capable of binding a target of choice.

The term “attach” or “attached” as used herein, refers to connecting or uniting by a bond, link, force or tie in order to keep two or more components together, which encompasses either direct or indirect attachment where, for example, a first molecule is directly bound to a second molecule or material, or one or more intermediate molecules are disposed between the first molecule and the second molecule or material. The term “bind”, “binding”, “conjugation” as used herein indicates an attractive interaction between two elements which results in a stable association of the element in which the elements are in close proximity to each other. If each element is comprised in a molecule the result of binding is typically formation of a molecular complex. Attractive interactions in the sense of the present disclosure includes both non-covalent binding and, covalent binding. Non-covalent binding as used herein indicates a type of chemical bond, such as protein-protein interaction, that does not involve the sharing of pairs of electrons, but rather involves more dispersed variations of electromagnetic interactions. Non-covalent bonding includes ionic bonds, hydrophobic interactions, electrostatic interactions, hydrogen bonds, and dipole-dipole bonds. Electrostatic interactions include association between two oppositely charged entities.

According to an exemplary embodiment, spectroscopic probes, such as methylene blue can be attached to an aptamer formed by an oligonucleotide through active ester coupling to an amine group (e.g. a 3′ C7 amine of an oligonucleotide aptamer) (see Examples section).

In particular, in several embodiments, a spectroscopic probe, and in particular a Raman probe, can be attached to an aptamer by a covalent bond, with or without one or more intermediate molecules, to any position where attachment does not interfere with target binding by the aptamer. For example in aptamer formed by an oligonucleotide probes can be directly bound to a covalent bond to the ribose, 2′-deoxyribose, or nitrogenous base of any nucleotides which comprise the aptamer In several embodiments, attachment is performed in proximity of or at one of the ends of the aptamer. For example in embodiments where the aptamer is formed by an oligonucleotide a spectroscopic probe can be attached to the 5′ terminus or the 3′ terminus depending on the probe. A skilled person will be able to verify a suitable location of the specific spectroscopic probe used. For example, verification can be performed by attaching the probe to the aptamer and verifying ability of the aptamer to bind a corresponding target before using the aptamer in an aptamer-based sensor herein described.

In aptamers-based sensor herein described attachment of the spectroscopic probe to a target binding aptamer is performed so that the detachment of the target binding aptamer from a suitable surface is monitored while maintaining its capability of target binding.

In particular, in several embodiments the aptamer is capable of specifically bind the target of interest. The wording “specific” “specifically” or “specificity” as used herein with reference to the binding of a first molecule to second molecule refers to the recognition, contact and formation of a stable complex between the first molecule and the second molecule, together with substantially less to no recognition, contact and formation of a stable complex between each of the first molecule and the second molecule with other molecules that may be present. Exemplary specific bindings are antibody-antigen interaction, cellular receptor-ligand interactions, polynucleotide hybridization, enzyme substrate interactions etc. The term “specific” as used herein with reference to a molecular component of a complex, refers to the unique association of that component to the specific complex which the component is part of. The term “specific” as used herein with reference to a sequence of a polynucleotide refers to the unique association of the sequence with a single polynucleotide which is the complementary sequence. By “stable complex” is meant a complex that is detectable and does not require any arbitrary level of stability, although greater stability is generally preferred.

In several embodiments, aptamers for specific target molecules suitable for inclusion in aptamer-based sensors herein described comprise aptamers that are either known or that can be derived through SELEX (Systematic Evolution of Ligands by Exponential Enrichment), also referred to as in vitro selection or in vitro evolution. In several embodiments aptamers are provided that have been developed and are able to specifically bind small molecules (nucleotides, cofactors, carbohydrates, and fluorescent dyes), antibodies (tetracyclines, streptomycin, aminoglycosides, chloramphenicol, and peptide antibiotic viomycin), proteins (VEGF, human interferon γ, angiopoietin-2, basic fibroblastic growth factor, platelet-derived growth factor, nucleic acid binding proteins, HIV-1, HIV reverse transcriptase, nuclear factor kappa B, hepatitis C virus-NS3, human neutrophil elastatse, thrombin, factor VIIa, factor IXa, cytotoxic T cell antigen, Prion protein PrP^{SC}), glucose and additional molecule substance or materials (including whole cells, tissues or portions thereof) are identifiable by a skilled person.

In several embodiments, aptamers of the aptamers-based sensors herein described comprise single-stranded (ss) oligonucleotides having an approximate length ≦100 nt or peptides (e.g. chemically synthesized), including, but not limited to, peptide aptamers comprised of a 20-amino acid variable region inserted into the active site of Escherichia coli thioredoxin A protein. Also, in several embodiments aptamers-based sensors herein described comprise aptamers capable of changing their secondary structures depending on ionic environments, on the presence of molecules having high affinity for the aptamers. An exemplary aptamer in this connection is provided by the thrombin binding aptamer illustrated in the examples section which forms ‘Guanine Quadruplex’ or ‘G-Quadruplex’ in the presence of a thrombin protein or high concentration of potassium (see Examples section).

Suitable surfaces for the aptamer based sensors herein described are formed by surface configured for detection through spectroscopy. Those surfaces comprise any space-confined, sharply edged or gapped structure in nano or sub-nano scale, which amplifies not only a surface Plasmon but also scattered light from a spectroscopic probe, (fluorescent or Raman probe). Typically, a surface Plasmon propagating on the surface is coupled at the structure, resulting in amplified local surface Plasmon resonance. In addition, the structure amplifies the scattered light for molecules near the structure. As a result, the structure induces molecular signal dramatically through the locally coupled surface Plasmon with a power of 4 (i.e. if surface plasmon is amplificated 10 times by the coupling effect, SERS or SERRS signal is amplified 10000 times).

The term “surface plasmon” as used herein indicates an electromagnetic wave along made of electron clouds, which propagates along a surface. Typically a surface plasmon has broad-band peaks corresponding to the electromagnetic wave resonating on the surface. The intensity of surface plasmon increases dramatically near the surface (<10 nm) and exponentially decrease with a distance from the surface. A strong surface plasmon can be exploited to amplify the intensity of incident light to molecule near the surface resulting in enhanced signals.

In particular in several embodiments, surfaces are prepared using a distribution of nanoparticles on a substrate. In particular the nanoparticles can be metal nanoparticles. The shape and size of the metal nanoparticles is selected in view of the desired enhancement because these factors influence the peak and amplitude of surface Plasmon. Not only spherical particles can be prepared but a variety of shapes including cubes, prisms, rods, octahedral, depending on reaction conditions and surface-active agents. A skilled person will be able to identify the desired shape and size of metal particles to be used in connection with a surface herein described using, for example, analytical model or numerical calculation. In several embodiments, the metal is formed by gold and/or silver nanoparticles that have surface plasmonic resonance peaks in visual wavelength range and the surfaces can be easily modified by conjugating functional chemicals. Additional nanoparticles comprise bimetal nanoparticles of noble metals such as silver and gold but also copper and/or platinum, mixed colloids of Ag or gold with catalytically important palladium and other nanoparticles described in U.S. Pat. No. 6,149,868 and in Ref. [37] each incorporated herein by reference in its entirety.

In particular, in several embodiments, the distribution of metal nanoparticles having a physical dimension smaller than 100 nanometers is provided on a suitable surface to form a metal nanostructure, which enables surface Plasmon to couple locally among the nanoparticles.

In several embodiments, distribution of metal on a suitable substrate results in a metal coated surface of a suitable substrate. The term “substrate” as used herein indicates an underlying support or substratum. Exemplary substrates include solid substrates, such as glass plates, microtiter well plates, magnetic beads, silicon wafers, silica nanosphere and additional substrates or surfaces identifiable by a skilled person upon reading of the present disclosure.

In several embodiments, metal nanoparticle can be distributed on a surface of the substrate using an aggregating agent (e.g. CuSO₄) so that locally coupled surface Plasmon on metal surface enhance Raman or fluorescence signal. In some embodiments, the substrate is modified by fabricating the necessary structure, and introducing on the substrate the sharply edged or small gap structure required for rendering the surface suitable for detection by the spectroscopic approach of choice. Additionally, treatment of the substrate surface can be performed to control the local plasmon surface in connection with the type of spectroscopy that is desired. For example, in some embodiments exemplified in the Examples section, aggregated nanoparticles (e.g. gold nanoparticles) can be used to create strong local surface Plasmon to induce surface enhanced Resonance Raman scattering. In some embodiments, single gold nanoparticle can be distributed to create moderate local surface Plasmon to induce surface enhance fluorescence. Exemplary suitable substrate include a gold or silver coated glass, polymer or ceramic slide, as well as Ag on silica nanospheres, roughened silver/gold surfaces, fabricated nano-pillars, other nanostructured metallic surfaces and additional suitable substrate identifiable by a skilled person.

In several embodiments, the aptamer of the aptamer based sensors herein described are adsorbed on a suitable surface. The term “adsorption” as used herein indicates accumulation of atoms or molecules on the surface of a material. Typically this process creates a film of the adsorbate (the molecules or atoms being accumulated) on the adsorbent's surface. In particular, in some embodiments, aptamers are physically adsorbed on a suitable surface wherein physical adsorption indicates a an accumulation of molecule on the surface that resembles the condensation of gases to liquids and depends on the physical, or van der Waals, force of attraction between the solid adsorbent and the adsorbate molecules and is characterized by no chemical specificity. In some embodiments aptamers are held to a solid surface by chemical forces that are specific for each surface and each aptamer. Chemical adsorption occurs usually at higher temperatures than those at which physical adsorption occurs; furthermore, chemical adsorption is ordinarily a slower process than physical adsorption and, like most chemical reactions, frequently involves an energy of activation.

In other embodiments, the aptamers are immobilized through electrostatic forces to a suitable surface and, in particular, to the plasmon surface of a suitable surface so that the spectroscopic probe attached to the aptamer is proximate to the plasmon surface. In other embodiments the aptamers can be connected to the surface by diffusing the aptamers in a solution located proximate to the surface. In some embodiments, the aptamers are immobilized on a surface through molecule such as mercaptobenzoic acid, covalently attached to a suitable surface (such as a silver surface) for pH sensing.

In several embodiments, the aptamer-based sensor herein described comprises an aptamer complex (a target-binding aptamer attaching a spectroscopic probe) and a metal nanomaterial inducing a surface Plasmon. In several of those embodiments, the target binding aptamer is conjugated to a Raman or fluorescent probe. In particular, in those embodiments the aptamer complex is capable of binding a corresponding target by exploiting the affinity of the aptamer and the binding is sensitively monitored by measuring enhanced signal of the probe with surface Plasmon resonating the probe molecule (e.g. through SERRS)

In several embodiments, aptasensors herein described can detect targets with a high sensitivity showing a limit of detection ≦100 pM and more particularly within a dynamic range spanning from about 100 pM to about 1 pM depending on the assay performed (see e.g. Example 2).

In several embodiments, aptasensors herein described can detect targets with high specificity, wherein the selective binding of the aptasensor with the intended target over other analytes can be shown by specific discrimination of the aptasensors in comparative binding assays (see e.g. Example 3).

In several embodiments, aptamers based sensor can detect targets, such as glucose, freely diffusing in a solution and/or in a sample. In some embodiments, aptamers based sensor can detect a target that is produced from a neighboring target producing material.

In several embodiments, aptasensors herein described are highly stable to the extent to enable detection of 1 nM target in presence of complex biofluids, such as 10% fetal calf serum, (see e.g. Example 4). In particular in some embodiments, stabilization can be achieved by chemically modifying the aptamers in particular at least one end thereof. For example, in embodiments wherein the aptamers are formed by oligonucleotides appropriate modifications comprise capping structure at 5′ and 3′ ends of aptamers and chemical modification of both ribose and phosphate backbone substitutes. Suitable techniques to perform such modifications comprise common synthetic strategies to cap 5′ end such as using a 5′-amino modifier phosphorammidite, PEG, cholesterol, fatty acid, polycations, and the like. Commonly used method for 3′ end modification of oligonucleotides is to attach an inverted thymidine to the 3′ end to create 3′-3′ linkage. Most commonly, the resistance of the backbone is improved with 2′-modified oligonucleotides by replacing with fluorophyrimidine, O-methyl, O-methylpyrimidine and the like. A skilled person will be able to identify additional techniques and related suitable modifications upon reading of the present disclosure.

In exemplary embodiments, where the aptasensors comprise peptide aptamers, stability of peptide aptamers can be enhanced by the incorporation of modified L-amino acids, incorporation of D-amino acids, disulfide bridging of cysteine residues, cyclization of the peptide, or covalent bonding or conjugation of the peptide aptamers to polymeric materials such as polyethylene glycol. However, since these techniques may also affect the ability of the peptide aptamer to bind to the target molecule, characterization of the stabilized peptide aptamer to ensure that it retains sufficient binding to the target molecule can be performed before using the peptide in aptamer based sensor in view of the experimental design of choice.

Aptasensors herein described can be used in a method to detect targets based on compared spectra of the spectroscopic surface of the aptasensors before and after contact with the target. The term “spectrum” as used herein indicates a representation (in particular a graphic representation such as a plot) of wavelengths reflected from a surface, which varies in function of chemical and/or physical properties (e.g. irregularities, atomic composition and/or molecular composition) of the surface.

In particular, in several embodiments, a first spectrum of the spectroscopic surface adsorbing the aptasensors herein described is detected.

In particular to this extent a nanostructured material capable of producing surface-enhanced effects through localized surface plasmons can be provided The nanostructure material can be formed by metal nanoparticles attached or adhered to a surface, or by fabricated structures.

Peptides or nucleic acid aptamers attaching Raman or fluorescent probes can then be provided in proximity of these nanostructured surfaces. In particular these aptamers can be attached covalently, electrostatically, or can be present diffusing in solution.

An excitation light is then applied to the sample (usually a laser, but not necessarily) and a corresponding spectrum can then be detected. In particular to this extent, a dispersive optical element (e.g. grating or prism) can be used in conjunction with a multi-pixel 2D or 1D detector to detect the spectrum. Also, the spectrum can be obtained by splitting the signal via frequency sensitive elements (such as dichroic mirrors and filters) and detected with more than one detecting element.

In an exemplary embodiment, an inverted microscope (Zeiss, Axiovert S100TV) was coupled with a helium-neon laser (LHRP-0501, 632.8 nm in wavelength, Research Electro Optics Inc., Boulder, Colo.), a spectrograph (SpectraPro 300i) equipped with a back-thinned LN-cooled CCD camera (Roper Scientific, Spec-10:100B (LN)) to collect spectra and a closed-loop X-Y piezo stage (Physik Instrumente) equipped with an avalanche photodiode (EG&G, SPCM-AQR-14), where both fluorescence and Raman scattering light were focused on through a 100-μm pinhole to take scanning image. The laser focused on and scattering from the SERRS substrate passed through a high numerical aperture oil immersion objective lens (Zeiss, Plan-Achromat 100X, N/A. 1.4). The spectra were analyzed by software (WinSpec/32, Roper Scientific) and the control of the stage and image analysis was done by software (Digital Instruments, Nanoscope IIIA). The SERRS spectra were measured with the laser of the power 10 μW and 10 s of integration time and the scanning was performed with laser power of 1 μW. The optical equipment was calibrated by measuring the Raman spectrum of a control sample (100% Toluene solution) before every experiment. To minimize the variation of signal with respect to the number of aggregated GNPs, the data were collected by shining the laser on GNP aggregates larger than the laser spot four times and averaging.

A second spectrum of the metal coated surface can then be detected following contact of the aptasensors with the intended target using an approach substantially similar to the ones used for detection of the first spectrum.

In particular, in an exemplary embodiment a protein (in TBB or serum) was introduced on the aptamer-immobilized SERRS substrate by dropping 10 μL and incubating for 1 hour at room temperature in the dark room. The displaced TBA and protein were subsequently removed via repeated rinsing five times with 10 μL 1×TBB. The experimental condition for measurement can be the same used for detecting the first spectrum.

The first and second spectrum are then compared and binding of the target to the target binding aptamer is qualitatively or quantitatively detected through based on the observed modifications of the second spectrum compared to the first spectrum. In particular, in some embodiments, target detection can be performed qualitatively by observing a difference between the first spectrum and the second spectrum. In some embodiments a quantitative detection can be performed by calculating a difference in the intensity between the first and the second spectrum using procedures identifiable by a skilled person.

In some embodiments, target detection is performed by detecting a first surface enhanced Raman spectrum of nanostructured novel metal (Au, Ag) surface physically adsorbing a target binding aptamer specific for the target and attaching a Raman probe. The method further comprises contacting the target binding aptamer on the metal surface with the target within an hour and at room temperature to allow the interaction of the target with the target binding aptamer. The method also comprises detecting a second surface enhanced Raman spectrum by the metal surface following contacting of the target binding aptamer with the target and comparing the first spectra and the second spectra.

In several embodiments, the sensing mechanism is based on the single-step target binding event to aptamer, which results in a decrease of the intensity of SERRS signals of the probe molecule attached to target binding aptamer (TBA) possibly due to the displacement of TBA from the metal coated surface, e.g. gold nanoparticle (GNP) surface.

In several embodiments, before the binding, an aptamer complex is immobilized on a metal nanomaterial with electrostatic force between an aptamer and a metal surface coated with charged molecules, which allows the probe to be proximate to the metal surface. In some of those embodiments the proximate to the metal surface where strong surface Plasmon is induced, the probe can emit strong Raman spectra enhanced by the surface Plasmon. Once the aptamer is bound to a target, the aptamer changes its secondary structure, causing weaker electrostatic binding to the metal surface and detachment of the complex from the metal surface. As a result, the signal of the probe decreases as the probe is removed away from the metal surface. The concentration of a target is quantitatively estimated by comparing signal change before and after the binding event.

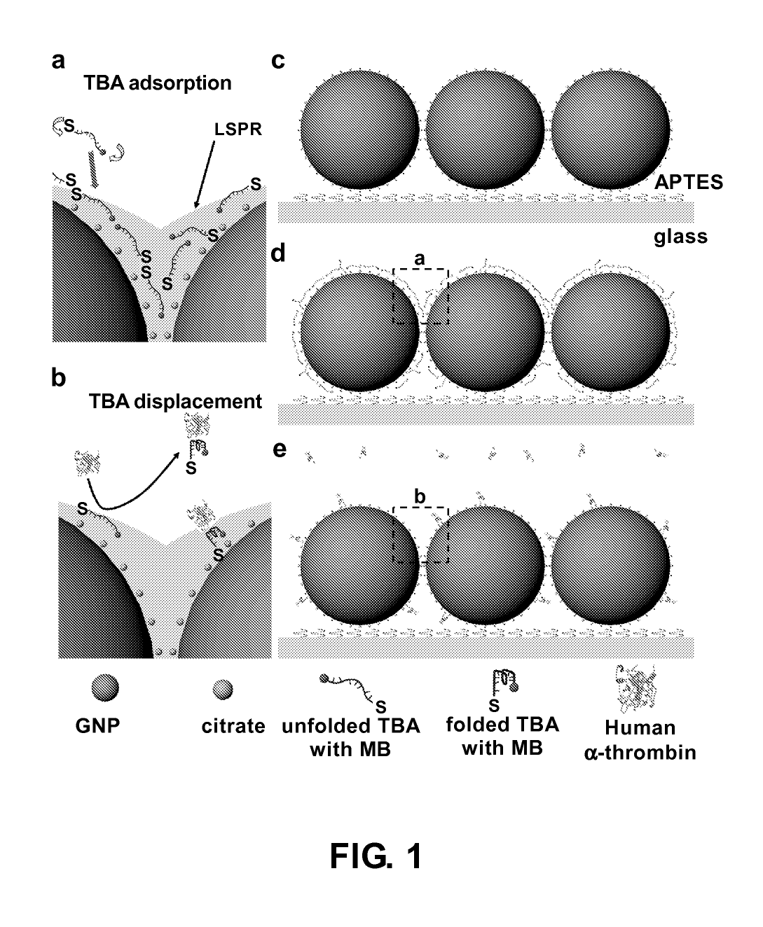

A schematic description of the detection mechanism according to some embodiments herein described is illustrated in FIG. 1. In particular, detection is performed based on the change of SERRS intensity of the Raman probe. In the absence of target, single-stranded TBA is unfolded and adsorbed onto a GNP surface this brings the methylene blue in close proximity to the GNP surface (FIGS. 1a and 1d), resulting in surface enhanced Raman scattering for methylene blue, Upon introduction of the target protein, TBA undergoes a conformational change (G-quadruplex formation) induced by a single-step binding event with thrombin (FIGS. 1b and 1e). TBA is thus being displaced from the GNP surface, hence reducing the intensity of the SERRS signal for MB.

Several embodiments take advantage of the ability of the SERRS technique to provide ultra sensitive detection of the probe molecules proximate to the GNP surface when a wavelength of incident light close to maximum of the probe molecule induces vibration resonance of the probe molecule and the surface plasmon resonance of gold. As a result, the described aptamer-based SERRS nanosensor allows much simpler detection scheme, coupled with a lower limit of detection when compared to other techniques based on multiple binding events or non-resonant SERS.

In particular, in several embodiments TBA displacement event is realized by exploiting the propensity of single-stranded DNA (ssDNA) to readily absorb onto GNP surfaces and to subsequently desorb upon its target binding. [Ref. 2,13,23,24] (See examples 2 and 3) In particular, in several embodiments, chemisorbed TBA, bound through a covalent gold-thiol bond, would remain immobilized upon addition/of sample solutions and provide a baseline signal level to verify the stability of the ssDNA aptamer and the light-sensitive Raman probe during exposure to complex biofluids, surface plasmon heating, and incident laser. Physisorbed TBA, in contrast, is held to the GNP surface only through electrostatic interactions between nitrogenous bases and the GNP surface and will be susceptible to displacement from the surface upon recognition and binding of target molecules.

In several embodiments, quantitative and sensitive detection of the target can be achieved/by monitoring the strong SERRS peaks of methylene blue, covalently attached to TBA, Methylene blue has/a So->S, transition centered at around 660 nm, allowing the incoming light in 632 nm wavelength to resonant vibration of the molecule, As a result, SERRS which is much stronger than non-resonant SERRS is observed while providing narrow spectral lines suited to quantitative analysis.

In several embodiments, the proposed detection method may be further implemented for multiplexed detection using different aptamer-based spectroscopic probe complexes. In those embodiments, detection of various targets of interest can be performed through specific detection of a corresponding spectroscopic probe attached to the aptamer that specifically bind one of those targets. For example, in embodiments where the probe is a Raman probe, detected spectroscopic Raman scattering corresponds to vibrational energy level (translation and rotation) in each functional group composing the matter and shows unique and several narrow band peaks. As a result, the Raman spectra provide unique information on molecular identity, orientation, conformation, and intermolecular interactions of the different aptamers adsorbed on the surface. Accordingly, in some embodiments aptamers detection performed through spectroscopy can be performed not only for label-free detection but also for multiplex detection, and even in samples wherein several targets of interest are included together with other analytes (e.g. biofluids or other complex samples).

In several embodiments, the aptamer-based sensor can be used for detection in conditions where protein or protein based sensor are usually degraded but aptamers are not (harsh condition). Those conditions include for example, detection from blood, where proteinases are present, which degrades protein-based antibodies in a short time. Also included is detection performed for a time and at a temperature that are usually associated with degradation of a protein which comprise exposure at high temperature for a short a amount of time or a lower temperature (e.g. room temperature) for a longer amount of time (e.g. several hours). High temperatures and low temperature are identifiable by a skilled person based on the specific protein. Modified aptamers are known to be stable even above melting temperature of several proteins.

Aptamers-based sensors herein described can be manufactured starting from the relevant components, according to various methods and procedures identifiable by a skilled person upon reading of the present disclosure.

In some embodiments, the aptamer-based nanosensor can be fabricated by a two-step procedure (see Example 1).

In a first step a metal distribution is provided on a suitable surface using techniques identifiable by a skilled person. For example a metal nanostructure can be formed on a suitable substrate and/or the substrate can be coated with metal nanoparticles.

In an exemplary embodiment, SERRS substrate can be fabricated by first fixing aggregated GNPs on a glass side, for example by incubating at room temperature for approximately 1 hour. The slide can be then thoroughly rinsed with appropriate solvents (e.g. acetone and isopropyl alcohol (IPA) sequentially) and then modified to fix the GNP on the slide (e.g. with amino-terminal group by immersion in APTES (10% v/v with IPA) for 10 minutes followed by rinsing with IPA and drying with N2 gas). A suitable amount (e.g. 14 μL mixture (50:1 v/v)) of GNP solution and aggregation agent, (e.g. 10 mM CuSO₄), can be spotted on the modified slide (e.g. 3 mm in diameter) that can be defined by a PDMS membrane and dried (e.g. at 60° C. for 40 minutes). Unfixed GNPs can then be removed by rinsing the substrate three times with 10 μL of DI water.

In a second step, one or more target binding aptamer attaching the spectroscopic probe is contacted to the metal coated surface to allow adsorption on the surface to attach the target binding aptamer to the surface.

In particular attachment of the target binding aptamer on a suitable surface can be performed so that the target binding aptamer is presented on the surfaces. The term “present” as used herein with reference to a compound or functional group indicates attachment performed to maintain the chemical reactivity of the compound or functional group as attached. Accordingly, a functional group presented on a molecule such as a functionalized membrane forming lipid, is able to perform under the appropriate conditions the one or more chemical reactions that chemically characterize the functional group.

For example, attachment of a target binding aptamer carrying a Raman probe on a metal coated surface can be performed in some embodiments by spontaneous coating of charged molecules (e.g. negatively charged citrate or positively charged Poly-L-Lysine) to a metal surface, followed by spontaneous immobilizing aptamer complexes to the metal surface. In some embodiments, the immobilization is improved by heating aptamer solution above the melting temperature of the aptamer.

In some embodiments, attachment of the aptamer on the surface can be performed by contacting a metal surface with a target binding aptamer attaching a spectroscopic probe for a time (e.g. within an hour) and under condition (e.g. above melting temperature of the aptamer) to allow physical adsorption of the target binding aptamer on the metal surface. The physical adsorption can be achieved by electrostatic force between metal surface treated with negatively charged molecule and positively charged nitrogeneous group of aptamer (or metal surface treated with positively charged molecule and negatively charged backbone of aptamer).

In an exemplary embodiment, where the spectroscopic probe is MB and the aptamer is a thrombin binding aptamer, both MB-tagged TBA and MB-tagged mutant TBA in a suitable buffer (e.g. 1×TBB) at a suitable concentration (e.g. 100 μM) can be immobilized on the GNP-substrate by incubating for a time and under condition that allows adsorption on a surface (e.g. overnight at room temperature in a dark room, or 1 hour at a temperature above melting temperature of the aptamer). The unbound aptamers can then be subsequently removed via repeated rinsing with a suitable buffer (e.g. five times with 10 μL 1×TBB).

In particular, in an exemplary embodiment the SERRS substrate was fabricated by first fixing aggregated GNPs on a glass side. The slide was thoroughly rinsed with acetone and isopropyl alcohol (IPA) sequentially and then modified with amino-terminal group by immersion in APTES (10% v/v with IPA) for 10 minutes followed by rinsing with IPA and drying with N₂ gas. A 14 μL mixture (50:1 v/v) of GNP solution and aggregation agent, 10 mM CuSO₄, was spotted on the modified slide (3 mm in diameter) defined by a PDMS membrane and dried at 60° C. for 40 minutes. Unfixed GNPs were removed by rinsing the substrate three times with 10 μL of DI water. Both MB-tagged TBA and MB-tagged mutant TBA in 1×TBB at the concentration of 100 μM were then immobilized on the GNP-substrate by incubating overnight at room temperature in a dark room. The unbound aptamers were subsequently removed via repeated rinsing five times with 10 μL 1×TBB.

According to some embodiments, the target binding aptamers, the Raman probes, the metal and/or the surface herein described can be provided in a system.

In particular, a system for detecting a target using aptasensor herein described can comprise at least two of the target binding aptamers, the Raman probes, the metal and/or the surface for combined simultaneous or sequential use in detecting the target according to a method herein described.

In several embodiments, the system includes in addition or in place of one or more of the above components a dispersive optical element (grating or prism) used in conjunction with a multi-pixel 2D or 1D detector to detect the spectrum. In those embodiments, spectrum can be obtained by splitting the signal via frequency sensitive elements (such as dichroic mirrors and filters) and detected with more than one detecting element.

The systems herein disclosed can be provided in the form of kits of parts. For example the target binding aptamers can be included as a molecule alone or already attached to a Raman probe.

In a kit of parts, the target binding aptamers, the Raman probes, and/or the metal are comprised in the kit independently possibly included in a composition together with suitable vehicle carrier or auxiliary agents. For example a target binding aptamers can be included in one or more compositions alone and/or included in a suitable vector. Also each of the Raman probes and/or metal can be included in a composition together with a suitable vehicle carrier or auxiliary agent. Furthermore, the Raman probes can be included in various forms suitable for appropriate conjugation with the target binding aptamers.

Additional components can also be included and comprise microfluidic chip, reference standards, and additional components identifiable by a skilled person upon reading of the present disclosure.

In the kit of parts herein disclosed, the components of the kit can be provided, with suitable instructions and other necessary reagents, in order to perform the methods here disclosed. In some embodiments, the kit can contain the compositions in separate containers. Instructions, for example written or audio instructions, on paper or electronic support such as tapes or CD-ROMs, for carrying out the assay, can also be included in the kit. The kit can also contain, depending on the particular method used, other packaged reagents and materials (such as wash buffers and the like).

In several embodiments, the SERRS aptasensors herein described and/or the related method and systems can be carried out in a microfluidic environment, such as the environment provided by the microfluidic platform described in the related U.S. patent application entitled “Microfluidic Platform and Related Methods and Systems” Ser. No. to be assigned filed on the same day of the present application with Docket No. IL12098, which is herein incorporated by reference in its entirety.

In particular in some of these embodiments, the aptasensors are included in an integrated platform together with cell trapping/culturing microfluidics to detect a target (such as a secreted growth factor) at physiological levels. In some of those embodiments, the microfluidic platform is composed of a microfluidic channel to provide cellular environments and a glass substrate coated or patterned with aptamer sensors, which are fabricated separately excluding an immobilization process for aptamer complex.

In particular, in several embodiments, the aptasensors herein described and related methods and systems can be operated in connection with an integrated microfluidic platform for applications such as cancer study and quantitatively detection of growth factors secreted from tumor cells. In several of those embodiments, the platform provides a dynamic tumor microenvironment by introducing various stimuli to isolated tumor calls with microfluidics and monitors the behavior by detecting the secretome with integrated nanosensors. The nanosensor is based on highly specific aptamer displacement upon target binding and the displacement monitoring with strong SERRS signals.

In some embodiment, the aptasensors are included in an integrated platform together with cell trapping/culturing microfluidics to detect the secreted growth factor at physiological levels. The microfluidic platform is composed of a microfluidic channel to provide cellular environments and a glass substrate coated or patterned with aptamer sensors, which are fabricated separately excluding an immobilization process for aptamer complex. Both of the microfluidic channel and the glass substrate are exposed to UV cleaning treatment to elevate surface energy and then contact for bonding. After leaving the bonded platform for an hour at room temperature, aptamer complex solution is introduced and incubated for an hour and then rinsed with buffer solution according to techniques identifiable by a skilled person upon reading of the present disclosure.

In several embodiments, the aptasensors comprised within a platform herein described can be a useful tool to quantitatively understand biomarker and related biological events such as the cancer-signaling pathway and discover drugs for cancer therapy.

Further details concerning the identification of the suitable aptamer-based sensor, and generally manufacturing and packaging of the aptasensors herein described, and of related systems can be identified by the person skilled in the art upon reading of the present disclosure.

### EXAMPLES

The methods and systems herein disclosed are further illustrated in the following examples, which are provided by way of illustration and are not intended to be limiting.

In the following examples, Applicants describe an exemplary aptamer-based surface enhanced resonance Raman scattering (SERRS) sensor with high sensitivity, specificity, and stability for the detection of a coagulation protein, human a-thrombin.

### Example 1

### SERRS Aptasensor Fabrication

An exemplary SERRS aptasensor was fabricated according to a procedure schematically illustrated in FIG. 1.

In the schematic illustration of FIG. 1, the SERRS-active substrate was fabricated by aggregating citrate-coated colloidal GNPs on Aminopropyltriethoxysilane-coated glass slides (FIG. 1c). After incubation with MB-conjugated TBA and extensive rinsing, the TBA-coated GNP substrate was measured with SERRS. Thrombin was subsequently titrated onto the TBA-GNP substrate, while monitoring the SERRS signal of MB (FIGS. 1a and 1d).

In particular, according to an exemplary procedure, the aptamer-based SERRS sensor was fabricated by a simple two-step procedure.

In a first step of the procedure, the SERRS-active substrate was fabricated by aggregating citrate-coated colloidal GNPs 80 nm in diameter on Aminopropyltriethoxysilane-coated glass slides.

In particular, a solution of gold nanoparticles (80 nm in diameter) was purchased from TED Pella, Inc., (Redding, Calif.) and PDMS membrane (HT 6240, 256 μm in thickness) was purchased from Rogers Corporation (Woodstock, Conn.). The colloidal GNPs were then aggregated on a glass cover slip previously treated with 3 aminopropyltriethoxysilane (APTES) (FIG. 1c). In particular, the slide was thoroughly rinsed with acetone and isopropyl alcohol (IPA) sequentially and then modified with amino-terminal group by immersion in APTES (10% v/v with IPA) for 10 minutes followed by rinsing with IPA and drying with N₂ gas. A 14 μL mixture (50:1 v/v) of GNP solution and aggregation agent, 10 mM CuSO₄, was spotted on the modified slide (3 mm in diameter) defined by a PDMS membrane and dried at 60° C. for 40 minutes. Unfixed GNPs were removed by rinsing the substrate three times with 10 μL of DI water. In this first part of the procedure, CuSO was used as an aggregation agent, since the charge on Cu″ induces aggregation of GNP covered with halide anions resulting in a fractal structure of GNPs. The aggregated GNPs presumably provide hot spots here a strong surface plasmon is resonated locally. The APTES-coated glass retains GNPs on the surface through harsh rinsing steps and also prevents any further aggregation of GNPs that may be caused by salt ions.

In a second step of the procedure, MB-conjugated TBA was introduced on the GNP-aggregated substrate at room temperature and allowed to incubate overnight in the dark (FIGS. 1b and 1d).

In particular, 3′ Methylene blue-tagged DNA oligonucleotides were synthesized by Biosearch Technologies, Inc. (Novato, Calif.), purified via RP-HPLC, and confirmed by mass spectroscopy to be >95% pure. The Thrombin Binding Aptamer (TBA) used was the following 5′-(CH2)6-S—S—(CH2)6-TAA GTT CAT CTC CCC GGT TGG TGT GGT TGG T-MB-3′ (SEQ ID NO:1). Methylene blue active ester was used for coupling to the amine group of the oligonucleotide (3′ C7 amine CPG purchased from Glen Research, Sterling, Va.). Human alpha-thrombin was purchased from Haematologic Technologies Inc. (Essex Junction, Vt.) in a powder form and was diluted with deionized water (DI water) to obtain desired stock concentration. 99% 3-Aminopropyltriethoxysilane (APTES), fetal calf serum and bovine serum albumin (BSA) were purchased from Sigma-Aldrich, Co. (St. Louis, Mo.). An appropriate amount of BSA was dissolved in DI water to obtain desired stock concentration.

The glass slide was then thoroughly rinsed with 1× thrombin binding buffer (TBB) 140 mM NaCl, 5 mM KCl, 1 mM MgCl2, 1 mM CaCl2, 20 mM Tris-acetate at pH 7.4.

The MB-tagged TBA in 1×TBB at the concentration of 100 μM was then immobilized on the GNP-substrate by incubating overnight at room temperature in a dark room. The unbound aptamers were subsequently removed via repeated rinsing five times with 10 μL 1×TBB. Protein (in TBB or serum) was introduced on the aptamer-immobilized SERRS substrate by dropping 10 μL and incubating for 1 hour at room temperature in the dark room. The displaced TBA and protein were subsequently removed via repeated rinsing five times with 10 μL 1×TBB.

After incubation with MB-conjugated TBA and extensive rinsing, the TBA-coated GNP substrate was measured with SERRS.

In particular, an inverted microscope (Zeiss, Axiovert S100TV) was coupled with a helium-neon laser (LHRP-0501, 632.8 nm in wavelength, Research Electro Optics Inc., Boulder, Colo.), a spectrograph (SpectraPro 300i) equipped with a back-thinned LN-cooled CCD camera (Roper Scientific, Spec-10:100B (LN)) to collect spectra and a closed-loop X-Y piezo stage (Physik Instrumente) equipped with an avalanche photodiode (EG&G, SPCM-AQR-14), where both fluorescence and Raman scattering light were focused on through a 100-μm pinhole to take scanning image. The laser focused on and scattering from the SERRS substrate passed through a high numerical aperture oil immersion objective lens (Zeiss, Plan-Achromat 100X, N/A. 1.4). The spectra were analyzed by software (WinSpec/32, Roper Scientific) and the control of the stage and image analysis was done by software (Digital Instruments, Nanoscope IIIA). The SERRS spectra were measured with the laser of the power 10 μW and 10 s of integration time and the scanning was performed with laser power of 1 μW. The optical equipment was calibrated by measuring the Raman spectrum of a control sample (100% Toluene solution) before every experiment. To minimize the variation of signal with respect to the number of aggregated GNPs, the data were collected by shining the laser on GNP aggregates larger than the laser spot four times and averaging.

### Example 2

### Target Detection Using SERRS Aptasensors

An aptasensor fabricated using the procedure exemplified in Example 1 was used to detect thrombin according to procedure schematically illustrated in FIG. 1e.

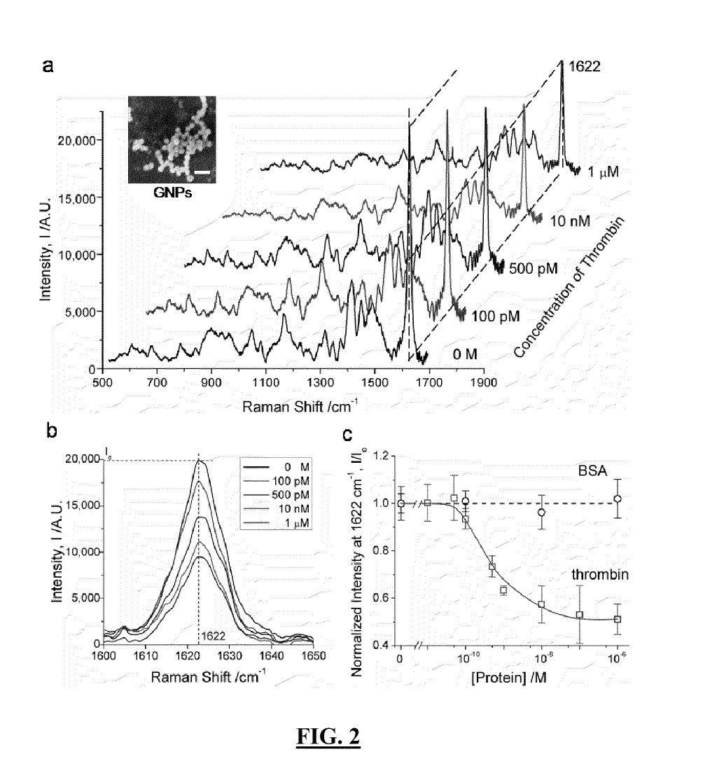

In the schematic illustration of FIGS. 1b and 1e, after through rinsing with 1× thrombin binding buffer (TBB) thrombin was introduced and incubated for one hour at room temperature in the dark (FIGS. 1b and 1e). The substrate was rinsed, kept in 1×TBB solution and covered with a cover glass and the SERRS spectra detected using the same procedure outlined in Example 1. The results are shown in FIG. 2.

In particular, in the illustration of FIG. 2 the spectra are shown that were collected from aggregated GNPs in a fractal structure (FIG. 2 inset, SEM image). The whole spectra are displayed after subtracting the presence of background from the original signal (FIG. 2a) for clarity and the most prominent Raman peak at 1622 cm⁻¹ (assigned to v(CC) ring and v(CNC) ring modes of methylene blue)3′ was compared for quantitative analysis (FIG. 2b, FIG. 2c).

In particular, in FIG. 2b the most prominent peak of MB, observed at 1622 cm⁻¹ is compared upon addition of varying concentrations of thrombin. In the illustration of FIG. 2c the SERRS signal drop is proportional to the thrombin concentration and reached a saturation level at 1 μM.

The effect of adding thrombin to the aptamer can clearly be observed through the reduction of the peak's intensity at the 1622 cm⁻¹. Initially, the peak's intensity dropped approximately 10% when introduced with 100 pM thrombin, However, with ˜1 pM thrombin concentration, the intensity dropped to about 50%, the observed saturation level (FIG. 2c). A limit of detection of 100 pM was then estimated for this assay with a dynamic range spanning 100 pM to 1 pM.

Therefore a decreased signal of MB SERRS was clearly distinguishable in the presence of thrombin (FIG. 2).

Accordingly, a detection mechanism herein described is based on the change of SERRS intensity of the Raman probe (FIG. 1). A possible interpretation that is provided herein for guidance and shall not be intended to be limiting, is that in absence of thrombin, single stranded TBA is unfolded and adsorbed onto a GNP surface. This brings the methylene blue in close proximity to the GNP surface (FIG. 1a), resulting in surface enhanced Raman scattering for methylene blue. Upon introduction of the target protein, TBA undergoes a conformational change (G-quadruplex formation) induced by a single-step binding event with thrombin (FIG. 1b). The interaction induces the displacement of thrombin binding aptamer (TBA) from gold nanoparticles (GNP) and the displacement is quantitatively monitored by measuring the SERRS signal change of a Raman-probe (methylene blue, ME) covalently attached to TBA.

### Example 3

### Specificity of Target Detection with SERRS Aptasensors

To verify that the signal change is dependent on the specific recognition of thrombin, a comparative detection performed using a nonspecific binding protein, bovine serum albumin (BSA).

The results are illustrated in FIG. 2c, in particular, when the substrate was treated with increasing concentration of BSA (up to 1 pM BSA), Applicants did not observe any significant change in the SERRS signal (FIG. 2c). This suggests that the TBA was not displaced from the GNP substrate, as TBA did not bind to BSA.

The specificity of the aptamer-based SERRS method was further investigated with a mutant TBA 5′-(CH₂)₆—S—S—(CH₂)₆-TAA GTT CAT CTC CCC AAT TGG TGT GGT TAA T-MB-3′ (SEQ ID NO: 2) (Mutant TBA) The nucleotides marked in bold indicate the position of essential “G” in the original aptamer sequence) in 1×TBB.

In particular, the 3′ Methylene blue-tagged Mutant TBA was synthesized by Biosearch Technologies, Inc. (Novato, Calif.), purified via RP-HPLC, and confirmed by mass spectroscopy to be >95% pure.

The MB-tagged mutant TBA in 1×TBB at the concentration of 100 μM was immobilized on the GNP-substrate by incubating overnight at room temperature in a dark room. The unbound protein/aptamers were subsequently removed via repeated rinsing five times with 10 μL 1×TBB. Protein (in TBB or serum) was introduced on the aptamer-immobilized SERRS substrate by dropping 10 μL and incubating for 1 hour at room temperature in the dark room. The displaced TBA and protein were subsequently removed via repeated rinsing five times with 10 μL 1×TBB.

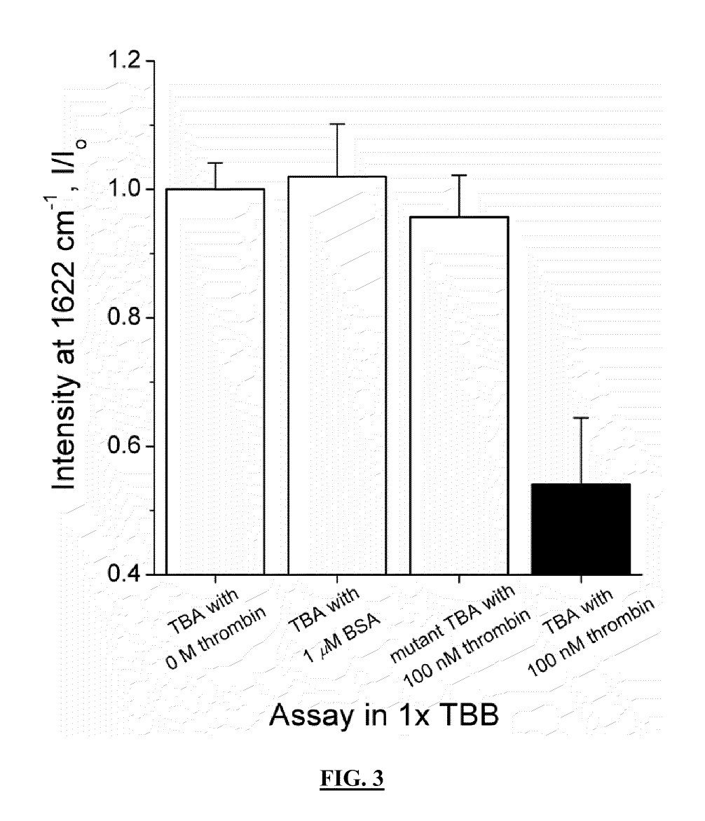

As shown in FIG. 3, upon the addition of 100 nM thrombin to the mutant TBA-GNP substrate, Applicants did not observe any decrease in the SERRS signal intensity.

Therefore, Applicants did not observe any appreciable decrease in the SERRS signal intensity from TBA in bovine serum albumin (BSA) solution and non-thrombin binding oligonucleotide (mutant TEA) in thrombin solution.

Taken together, these observations suggest that the displacement mechanism relies on specific recognition and that the aptamer-based SERRS method is highly specific for the detection of TBA-thrombin interaction.

### Example 4

### Stability of the SERRS Aptasensor

The stability of the nanosensor was evaluated in the presence of complex mixtures including nucleases, i.e. serum.

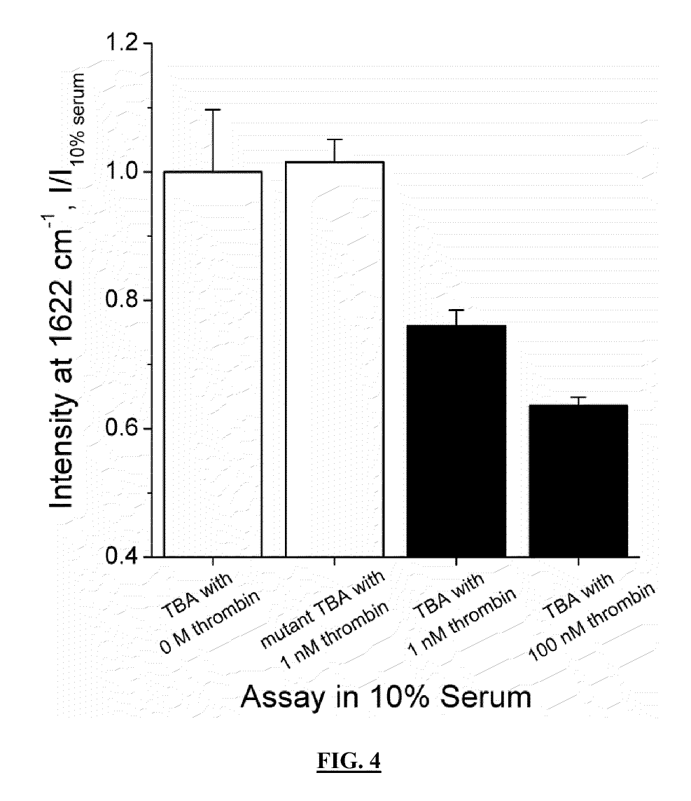

In particular, assays were performed on aptasensors fabricated as exemplified in Example 1, in 10% fetal calf serum (v/v in 1× thrombin binding buffer) and the results illustrated in FIG. 4.

Applicants observed −20% signal decrease when the substrate was treated with 10 fetal calf serum (v/v in TBB), indicating that the TBA was minimally displaced from the aggregated GNPs by the serum's constituents.

However, based on a “serum baseline at 10% serum,” the signal dropped to −75% with the addition of 1 nM thrombin in 10% serum, while the signal dropped to a saturating level of 60% when the input thrombin was increased to 100 nM. Mutant TBA-GNP substrate was unaffected with the input of 1 nM thrombin.

Increasing the concentration of calf serum utilized Applicants noticed that the SERRS signal drop reached the 50% saturation in 75% serum. Applicants hypothesize that the displacement of TBA in elevated serum levels was probably due to stronger electrostatic interference by serum's salt contents rather than by nuclease activity in the serum, which are unlikely to degrade the immobilized, 5′- and; 3′ capped TBA35. With this “serum baseline,” the intensity in the presence of 10% serum, Applicants proceeded to introduce thrombin to both the TBA-GNP substrate and the mutant TBA-GNP substrate. With the addition of the thrombin in 10% serum to the TBA-GNP substrate, the signal dropped −25% from the serum baseline, and the signal further decreased to a saturating level of 60% when the thrombin concentration reached 100 nM. In contrast, the mutant TBA-GNP substrate did not produce any significant signal when exposed to 1 nM thrombin in 10% serum.

These observations collectively indicate that the aptasensor can perform in presence of 10% serum. Applicants also note that mutant TBA-GNP substrate is unaffected by nucleases in serum. Thus, the mutant aptamer both effectively and conveniently provides a “negative control” that enables specific protein detection for each respective assay performed in 10% serum.

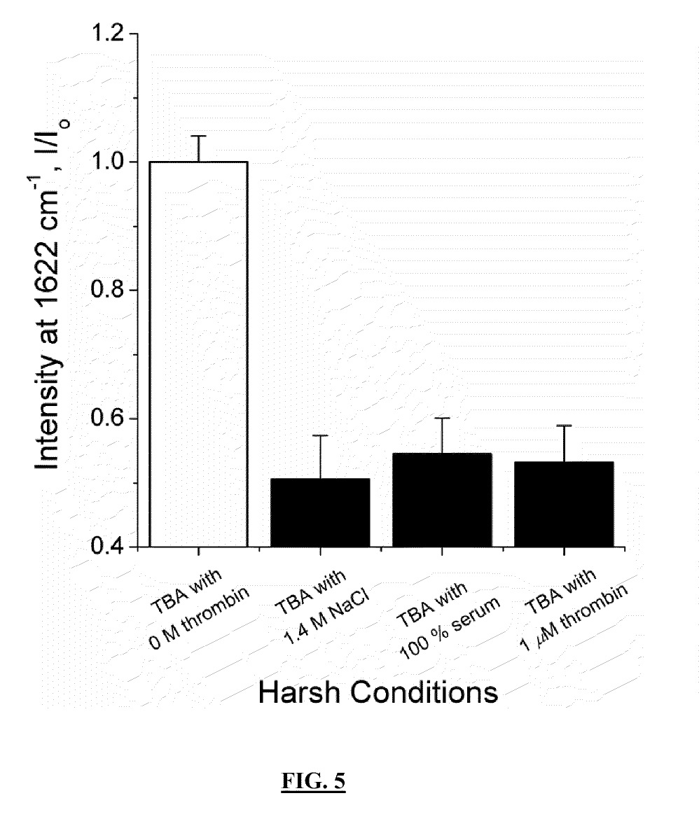

To further test stability of the aptasensors, Applicants performed assays in harsh conditions (high NaCl, serum, and thrombin). The results illustrated in FIG. 5 show an evaluation of 50% signal saturation. In particular, the relative intensity of most prominent signal afforded by MB, measured at 1624 cm⁻¹, decreased to a saturating level ˜50% under the harsh conditions: high NaCl, serum, and thrombin indicating that physisorbed TBA was removed by ion effects or thrombin-interaction.

In conclusion, the above results demonstrate highly sensitive, selective, and stable aptamer-based SERRS nanosensor for the detection of a target exemplified by throming thrombin. In particular, in view of the above results, the limit of detection of the aptamer-based sensor is 100 pM (below the dissociation constant of TBA, 25 nM).

A possible interpretation that is provided herein only for guidance and is not intended to be limiting is that this low limit of detection is possible because the aptamer-based SERRS signal depends on the number of aptamers displaced from the surface, not the number of aptamers that are bound to target. In addition, this assay is sufficiently stable to identify the target in the presence of 10% serum. Utility of this method is highly flexible as it can easily accommodate numerous protein specific aptamers, each conjugated to a unique Raman probe, in a single platform, thus enabling multiplex protein detection.

Given the size and simplicity of the proposed detection schematic, this method can be readily used in a miniaturized and integrated platform as it can easily accommodate numerous protein-specific aptamers with a variety of Raman probes for high throughput and multiplexed drug screening and biomedical diagnostics

The examples set forth above are provided to give those of ordinary skill in the art a complete disclosure and description of how to make and use the embodiments of the aptamer based sensors, systems and methods of the disclosure, and are not intended to limit the scope of what the inventors regard as their disclosure. Modifications of the above-described modes for carrying out the disclosure that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

The entire disclosure of each document cited (including patents, patent applications, journal articles, abstracts, laboratory manuals, books, or other disclosures) in the Background, Summary, Detailed Description, and Examples is hereby incorporated herein by reference.

It is to be understood that the disclosures are not limited to particular compositions or biological systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

Although any methods and materials similar or equivalent to those described herein can be used in the practice for testing aptamers based sensor and related systems and methods of the disclosure, specific examples of appropriate materials and methods are described herein for guidance purpose.

A number of embodiments of the disclosure have been described. Nevertheless, it will be understood that various modifications may be made without departing from the spirit and scope of the present disclosure. Accordingly, other embodiments are within the scope of the following claims.

### REFERENCES

1. Ellington, A. D.; Szostak, J. W. Nature 1990, 346, 818-822
2. Tuerk, C.; Gold, L. Science 1990, 249, 505-510
3. Jayasena, S. D. Clin. Chem. 1999, 45, 1628-1650.
4. Sullenger, B. A.; Gilboa, E. Nat. Rev. 2002, 418, 252-258
5. Pestourie, C.; Tavitian, B.; Duconge, F. Biochimie 2005, 87, 921-930
6. Bunka, D. H. J.; Stockley, P. G. Nat. Rev. Microbio. 2006, 4, 588-596
7. Klussmann, S. The Aptamer Handbook: Functional Oligonucleotides and Their Applications; WILEY-VCH Verlag GmbH & Co. KGaA: New York, 2006.
8. Fischer, N. O.; Tarasow, T. M.; Tok, J. B. H. Curr. Opin. Chem. Biol. 2007, 11, 316-328
9. Szakacs, G.; Paterson, J. K.; Ludwig, J. A.; Booth-Genthe, C.; Gottesman, M. M. Nat. Rev. Drug Discovery 2006, 5, 219-234
10. Xiao, Y.; Piorek, B. D.; Plaxco, K. W.; Heeger, A. J. J. Am. Chem. Soc. 2005, 127, 17990-17991
11. Baker, B. R.; Lai, R. Y.; Wood, M. S.; Doctor, E. H.; Heeger, A. J.; Plaxco, K. W. J. Am. Chem. Soc. 2006, 128, 3138-3139
12. Stojanovic, M. N.; Landry, D. W. J. Am. Chem. Soc. 2002, 124, 9678-9679
13. Huang, C. C.; Huang, Y. F.; Cao, Z.; Tan, W.; Chang, H. T. Anal. Chem. 2005, 77, 5735-5741
14. Liu, J.; Lu, Y. Nat. Protocols 2006, 1, 246-252
15. Bagalkot, V.; Zhang, L.; Levy-Nissenbaum, E.; Jon, S.; Kantoff, P. W.; Langer, R.; Farokhzad, O. C. Nano Lett. 2007, 7, 3065-3070
16. Xiao, Y.; Lubin, A. A.; Heeger, A. J.; Plaxco, K. W. Angew. Chem. Int. Ed. 2005, 44, 5456-5459
17. Radi, A. E.; AceroSanchez, J. L.; Baldrich, E.; O'Sullivan, C. K. J. Am. Chem. Soc. 2006, 128, 117-124
18. Wei, H.; Li, B.; Li, J.; Wang, E.; Dong, S. Chem. Commun. 2007, 3735-3737
19. Fang, X.; Sen, A.; Vicens, M.; Tan, W. ChemBioChem 2003, 4, 829-834
20. Shlyahovsky, B.; Li, D.; Weizmann, Y.; Nowarski, R.; Kotler, M.; Willner, I. J. Am. Chem. Soc. 2007, 129, 3814-3815
21. Wang, Y.; Wei, H.; Li, B.; Ren, W.; Guo, S.; Dong, S.; Wang, E. Chem. Commun. 2007, 5220-5222
22. Chen, J.-W.; Liu, X.-P.; Feng, K.-J.; Liang, Y.; Jiang, J.-H.; Shen, G.-L.; Yu, R.-Q. Biosens. Bioelectron 2008, 24, 66-71
23. Li, H.; Rothberg, L. Proc. Natl. Acad. Sci. U.S.A. 2004, 101, 14036-14039
24. Li, H.; Rothberg, L. J. Anal. Chem. 2004, 76, 5414-5417
25. Bock, L. C.; Griffin, L. C.; Latham, J. A.; Vermaas, E. H.; Toole, J. J. Nature 1992, 355, 564-566
26. Vairamani, M.; Gross, M. L. J. Am. Chem. Soc. 2003, 125, 42-43
27. Yau, H. C. M.; Chan, H. L.; Sui, S. f.; Yang, M. Thin Solid Films 2002, 413, 218-223
28. Herne, T. M.; Tarlov, M. J. J. Am. Chem. Soc. 1997, 119, 8916-8920
29. Gearheart, L. A.; Ploehn, H. J.; Murphy, C. J. J. Phys. Chem. B 2001, 105, 12609-12615
30. Nicolai, S. H. A.; Rubim, J. C. Langmuir 2003, 19, 4291-4294
31. Stokes, R. J.; Macaskill, A.; Lundahl, P. J.; Smith, W. E.; Faulds, K.; Graham, D. Small 2007, 3, 1593-1601
32. Bell, S. E. J.; Sirimuthu, N. M. S. J. Phys. Chem. 2005, 109, 7405-7410
33. Weitz, D. A.; Oliveria, M. Phys. Rev. Lett. 1984, 52, 1433
34. Choi, D.; Kang, T. K.; Cho, H.; Choi, Y.; Lee, L. P. Lab on a Chip 2009, 9, 239-243
35. Nicolai, S. H. d. A.; Rodrigues, P. R. P.; Agostinho, S. M. L.; Rubim, J. C. J. Electroanal. Chem. 2002, 527, 103-111
36. Ruckman, J.; Green, L. S.; Beeson, J.; Waugh, S.; Gillette, W. L.; Henninger, D. D.; Claesson-Welsh, L.; Janjic, N. J. Biol. Chem. 1998, 273, 20556-20567
37. Hering Katharina Clalla Dana, Ackermann Katrin Dörfer Thomas, Möller Robert, Schneidewind Henrik, Mattheis Roland, Fritzsche Wolfgang, Rösch Petra, Popp Jürgen in Anal Bioanal Chem (2008) 390:113-124

What is claimed is:

1. An aptamer based-sensor comprising:
a target binding aptamer attaching a spectroscopic probe, and
a surface configured for detection through spectroscopy;
wherein the target binding aptamer attaching the spectroscopic probe is capable of binding a corresponding target, and
wherein the target binding aptamer attaching the spectroscopic probe is detectable through surface enhanced spectroscopy of the surface.

2. The aptamer based sensor of claim 1, wherein the target binding aptamer is a single-stranded oligonucleotide having an approximate length ≦100 nt or a peptides having an approximate length of 20 amino acids.

3. The aptamer based sensor of claim 1, wherein the Raman probe is selected from the group consisting of trans-1,2 bis-(4-pyridyl)ethylene (BPE), Cy-3, Cy-3.5, Cy-5, Cy-5.5, Cy-7, Rhodamine 6G (R6G), methylene blue (MB), FAM, TAMRA, TET, ROX, Yakima yellow, and BODIPY TR-X.

4. The aptamer based sensor of claim 1, wherein the target binding aptamer is conjugated to the Raman probe through covalent linkage.

5. The aptamer based sensor of claim 1, wherein the metal coated surface is formed by a metal coated surface of a substrate selected from the group consisting of a glass plate, a polymer plate or a ceramic plate.

6. The aptamer based sensor of claim 1, wherein the metal is selected from the group comprising gold nanoparticles and silver nanoparticles.

7. The aptamer based sensor of claim 1, wherein the target is a nucleotide, a cofactor, a carbohydrate, a fluorescent dye, VEGF, human interferon γ, angiopoietin-2, basic fibroblastic growth factor, platelet-derived growth factor, nucleic acid binding proteins, HIV-1, HIV reverse transcriptase, nuclear factor kappa B, hepatitis C virus-NS3, human neutrophil elastatse, thrombin, factor VIIa, factor IXa, cytotoxic T cell antigen, Prion protein PrP^{SC}.

8. A method to detect a target, the method comprising
detecting a first surface enhanced spectrum of a surface configured for detection through spectroscopic analysis, the surface presenting a target binding aptamer, with the target binding aptamer specific for the target and attaching a spectroscopic probe;
contacting the target binding aptamer presented on the surface with the target for a time and under conditions to allow binding of the target with the target binding aptamer;
detecting a second surface enhanced spectrum of the metal surface following contacting of the target binding aptamer with the target, and
comparing the first spectrum and the second spectrum.

9. A system to detect a target, the system comprising
at least two of a target binding aptamer, a spectroscopic probe and a surface configured for detection through spectroscopic analysis,
the target binding aptamer, the spectroscopic probe and surface for combined simultaneous or sequential use in the method of claim 8.

10. The system of claim 9, the system further comprising a dispersive optical element used in conjunction with a multi-pixel 2D or 1D detector to be used to detect the first and or the second spectrum.

11. A method to manufacture an aptamer based sensor, the method comprising:
contacting a surface configured for detection through spectroscopic analysis with a target binding aptamer attaching a spectroscopic probe for a time and under condition to allow adsorption of the target binding aptamer on the coated metal surface.

12. The method of claim 11, wherein contacting is performed to allow physical adsorption of the aptamer on the surface.


SEQUENCE LISTING (2 sequences)

SEQ ID NO: 1 - DNA, 31 bp, Artificial Sequence
  Synthetic Polynucleotide
taagttcatctccccggttggtgtggttggt

SEQ ID NO: 2 - DNA, 31 bp, Artificial Sequence
  Synthetic Polynucleotide
taagttcatctccccaattggtgtggttaat